(19) United States
(12) Patent Application Publication
(10) Pub. No.: US 2007/0078493 A1
(43) Pub. Date: Apr. 5, 2007

(54) Impedance-based penile tumescence sensor

(75) Inventor: Martin T. Gerber, Maple Grove, MN (US)

Correspondence Address: SHUMAKER & SIEFFERT, P. A., 8425 SEASONS PARKWAY, SUITE 105, ST. PAUL, MN, 55125, US

(73) Assignee: Medtronic, Inc., Minneapolis, MN (US)

(21) Appl. No.: 11/243,259

(22) Filed: Oct. 4, 2005

(51) Int. Cl. A61N 1/00

(52) U.S. Cl. 607/39; 607/143

(57) ABSTRACT

The disclosure describes techniques for sensing penile tumescence based on the impedance of tissue between two electrodes implanted in the penis, which may vary based on penile tumescence, e.g., penile length or circumference. One of the electrodes emits an electrical signal, and the other of the electrodes detects the emitted electrical signal. Information relating to the detected electrical signal, e.g., information indicating the degree of penile tumescence, may be used in a therapeutic penile tumescence control system. For example, such information may be used to control delivery of electrical stimulation of therapeutic substances to the pelvic floor nerves that stimulate erections. In addition, such information may be recorded and presented a physician.

### TECHNICAL FIELD

The invention relates to implantable medical devices and, more particularly, implantable sensors.

### BACKGROUND

Sexual dysfunction of the penis is a common problem afflicting males of all ages, genders, and races. Erectile dysfunction is a serious condition for many men, and it may include a variety of problems. Some of these problems include the inability to create an erection, incomplete erections and brief erectile periods. These conditions may be associated with nervous system disorders, and may be caused by aging, injury, or illness.

In some cases, erectile dysfunction can be attributed to improper nerve activity that incompletely stimulates the penis. For example, stimulation from the brain during arousal and sexual activity is responsible for activating an erection. With respect to erectile disorders, the problem may be a lack of sufficient stimulation from the brain, or a break in communication of the stimulation. Erectile disorders may additionally or alternatively involve dysfunctional parasympathetic function that can be attributed to many factors including illness or injury.

Methods for treating erectile dysfunction include pharmaceutical treatment and electrical stimulation. Delivery of electrical stimulation to nerves running through the pelvic floor may provide an effective therapy for many patients. For example, an implantable stimulator may be provided to deliver electrical stimulation to the pudendal or cavernous nerves to induce an erection.

### SUMMARY

The disclosure is directed to techniques for sensing penile tumescence. More particularly, a system according to the invention includes electrodes implanted at respective locations within a penis to sense the impedance of tissue between the electrodes. The impedance between the electrodes varies based on the distance between each electrode, which varies based on the degree of tumescence of the penis, e.g., penile length or diameter.

Systems including such electrodes and employing the described techniques may provide short- or long-term monitoring of penile tumescence for storage and offline analysis by a physician. For example, systems according to the invention may include an external programmer that receives impedance/tumescence information from an implantable device. The external programmer may present impedance/tumescence information to a user, such as a physician.

Additionally or alternatively, a system according to the invention may include an implantable medical device that delivers therapy, e.g., electrical or chemical stimulation therapy, based on the impedance/tumescence information. In this manner, the impedance/tumescence information may provide feedback in a closed-loop therapy delivery system to control and sustain a state of erection during the course of sexual activity, thus treating sexual dysfunction or, more specifically, erectile dysfunction. The implantable medical device may directly receive impedance/tumescence information, or may be controlled by an external programmer that receives impedance/tumescence information.

In some embodiments, one or both of the electrodes used to sense penile tumescence may be coupled to a therapy-delivering implantable medical device. In other embodiments, one or both of the electrodes are coupled to one or more implantable modules capable of wirelessly communicating with an implantable medical device and/or the external programmer. The implantable modules may be implanted in the penis. In embodiments that include one or more implantable modules and a therapy-delivering implantable medical device, the implantable modules may be separate from the implantable medical device

In one embodiment, the invention is directed to a method comprising emitting an electrical signal from a first electrode implanted within a penis of a patient at a first location, detecting the electrical signal via a second electrode implanted within the patient at a second location, and delivering therapy from an implantable medical device to the patient based on the electrical signal to control penile tumescence.

In another embodiment, the invention is directed to a system comprising a first electrode implanted within a penis at a first location that emits an electrical signal, a second electrode implanted within the penis at a second location that detects the electrical signal, and an implantable medical device to that delivers therapy to the patient based on the detected electrical signal to control penile tumescence.

In an additional embodiment, the invention is directed to a system comprising a first electrode implanted within the penis at a first location to emit an electrical signal, a second electrode implanted within the penis at a second location, and an implantable sensor comprising a housing implanted within the penis. The implantable sensor is coupled to at least the second electrode, detects the electrical signal via the second electrode, and comprises telemetry circuitry that wirelessly transmits information relating to the detected electrical signal. The transmitted information indicates the degree of tumescence of the penis.

In various embodiments, the invention may provide one or more advantages. For example, implanting electrodes within the penis permits changes in impedance and tumescence to be accurately detected, and saved for review or used in real-time to provide closed-loop feedback therapy. Using systems according to the invention, tumescence can be sensed without significantly obstructing or altering the physiological function or the penis. For example, the electrodes and/or small modules used with some embodiments of the invention may be sized and/or positioned such that they do not interfere with normal sexual activity. Additionally, such electrodes and/or small modules may be implanted within the penis with minimally invasive surgical procedures.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a schematic diagram illustrating an example system that includes an implantable medical device that senses penile tumescence and delivers a therapy to a patient as a function of the sensed tumescence.

FIG. 2 is a schematic diagram further illustrating the example system of FIG. 1.

FIG. 3 is a schematic diagram illustrating another example system that senses penile tumescence, the system including a separate implantable tumescence sensor.

FIG. 4 is a schematic diagram further illustrating the example system of FIG. 4.

FIG. 5 is an enlarged, cross-sectional side view of the implantable sensor of FIGS. 4 and 5.

FIG. 6 is a schematic diagram illustrating implantation of the implantable sensor of FIGS. 4 and 5 within the penis.

FIG. 7 is a schematic diagram illustrating another example system that senses penile tumescence, the system including two separate implantable modules.

FIG. 8 is a functional block diagram illustrating various components of the implantable medical device of FIGS. 1 and 2.

FIG. 9 is functional block diagram illustrating various components of the implantable sensor of FIGS. 4 and 5.

FIG. 10 is a functional block diagram illustrating various components of the implantable medical device of FIGS. 4 and 5.

FIG. 11 is a flow chart illustrating a technique for delivery of stimulation therapy to alleviate sexual dysfunction based on closed loop feedback from an implantable tumescence sensor.

### DETAILED DESCRIPTION

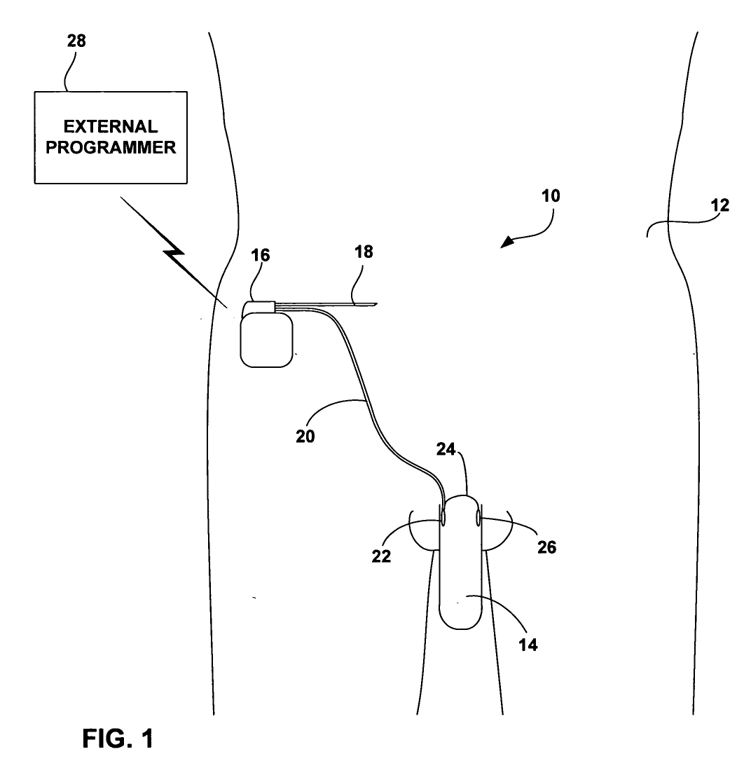

FIG. 1 is a schematic diagram illustrating an example system 10 that includes an implantable medical device (IMD) 16 that senses the degree of tumescence of a penis 14 of a patient 12, e.g., penile tumescence. In the illustrated embodiment, IMD 16 delivers a therapy to patient 12 as a function of the sensed penile tumescence. IMD 16 may deliver the therapy to control the tumescence of penis to, for example, alleviate sexual or erectile dysfunction. The therapy delivered by IMD 16 may include, as examples, one or more of electrical stimulation or delivery of a substance, such as a pharmaceutical, chemical or genetic substance.

In the illustrated example, IMD 16 is coupled to a lead 18 for delivery of electrical stimulation as therapy. IMD 16 may, for example, deliver stimulation via lead 18 to one or more nerves associated with the erectile functioning of penis 14, such as the prostate parasympathetic nerves, cavernous nerves, pudendal nerves, or sacral nerves. In other embodiments, IMD 16 may additionally or alternatively deliver one or more substances via a catheter to such nerves, or to the tissues of penis 14.

In addition to IMD 16 and lead 18, system 10 includes a first electrode 22 implanted at a first location within penis 14, and a second electrode 26 implanted at a second location within penis 14. A sensing lead 20 couples IMD 16 to electrode 22, and couples IMD 16 to electrode 26 via a penile lead 24. Leads 20 and 24 may be, for example, subcutaneously tunneled from IMD 16 to first and second locations.

IMD 16 drives electrode 22 to emit an electrical signal, and detects the electrical signal via second electrode 26. The detected electrical signal will vary as a function of the tumescence of penis 14. More particularly, the impedance of penile tissue between electrodes 22, 26 will vary as a function of the tumescence of penis 14. Consequently, IMD 16 may sense penile tumescence based on one or more of the voltage or current of the detected electrical signal. In other embodiments, electrode 26 may emit the electrical signal, while electrode 22 detects the electrical signal.

IMD 16 may, but need not, actually determine the impedance of the penile tissue based on the detected electrical signal. IMD 16 may detect impedance and penile tumescence by comparing the voltage and/or current of the detected electrical signal to that of the emitted electrical signal. IMD 16 may additionally or alternatively detect changes impedance and penile tumescence by comparing a current voltage and/or current of the detected electrical signal to previously measured voltages and/or currents of the detected electrical signal.

In any event, IMD 16 may deliver therapy to patient 12 based on the detected electrical signal by delivering the therapy based on one or more of voltage, current or impedance, or changes therein. For example, IMD 16 may initiate or terminate electrical stimulation based on the voltage, current or impedance, or changes therein, reaching a threshold value. IMD 16 may additionally or alternatively vary electrical stimulation parameters, e.g., amplitude, pulse width or rate, based on the voltage, current or impedance, or changes therein, to provide a closed-loop feedback of erectile state information during the course of sexual activity. In other embodiments, the rate of substance, e.g., drug, delivery, or the type of substances delivered, may be controlled by IMD 16 based on the one or more of voltage, current or impedance, or changes therein. For example, erectile function may be effectively controlled through the balancing of two delivered drugs as a function of the one or more of voltage, current or impedance, or changes therein.

In some embodiments, IMD 16 may generate adjustments to electrical stimulation parameters based on the detected electrical signal to provide electrical stimulation that supports distinct phases of sexual activity, and transition between such phases. For example, based on detected electrical signal, IMD 16 may adjust stimulation parameters to maintain a particular phase of sexual activity, transition from one phase to another, and transition from one phase to a cessation of sexual activity. Examples of distinct phases of sexual activity include arousal, e.g., desire, erection or lubrication, and orgasm or ejaculation. To support distinct phases of sexual activity and progression between phases, IMD 16 may be configured to operate in accordance with the techniques described in U.S. patent application Ser. No. 10/441,784, to Martin Gerber, filed May 19, 2003, entitled “TREATMENT OF SEXUAL DYSFUNCTION BY NEUROSTIMULATION,” the entire content of which is incorporated herein by reference.

In the illustrated embodiment, system 10 also includes an external programmer 28. Patient 12 may control the therapy delivered by IMD 16 via programmer 28. For example, patient 12 may initiate or terminate delivery of therapy by IMD 16 via programmer 28.

In some embodiments, external programmer 28 may control delivery of therapy by IMD 16 based on degree of penile tumescence sensed by IMD 16, e.g., the electrical signal detected by IMD 16. For example, rather then itself initiating, terminating or adjusting the therapy based on the one or more of voltage, current or impedance, IMD 16 may transmit the voltage, current or impedance to programmer 28, which may transmit commands to IMD 16 to initiate, terminate or adjust the delivery of therapy based on the voltage, current or impedance.

Further, in some embodiments, in addition to controlling therapy based on the voltage, current or impedance, IMD 16 or programmer 28 may control therapy based on input received from the patient. The input programmer 28 receives from the patient may indicate the degree or intensity of pleasure or other sensations experienced by the patient, or whether the patient is experiencing pain.

Additionally, one or both of IMD 16 and external programmer 28 may store the sensed voltages, currents or impedances, and thereby store indications of penile tumescence over time for short or long-term monitoring of penile tumescence. In either case, such penile tumescence information may be presented to a user, such as a physician, via external programmer 28, or another computing device. Further, the invention is not limited to embodiments that include a therapy delivering IMD. In some embodiments, an IMD 16 and/or external programmer 28 may store sensed penile tumescence information without delivery of therapy, for short or long-term monitoring of penile tumescence.

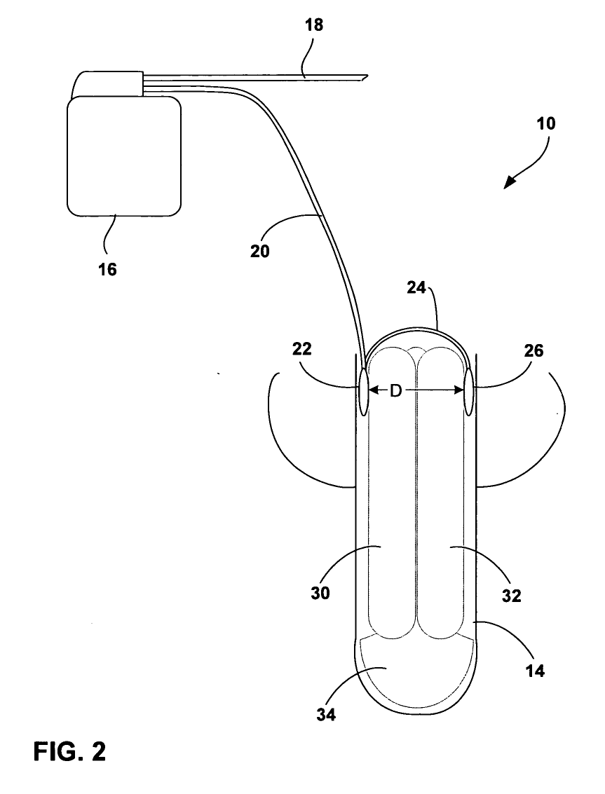

FIG. 2 is schematic diagram further illustrating system 10. In the example shown in FIG. 2, first electrode 22 is implanted near the base of the shaft of penis 14 on a first side, while second electrode 26 is implanted near the base of the shaft of penis 14 on a second side, e.g., approximately opposite electrode 22. An electrical signal emitted by electrode 22 travels approximately the distance D between both electrodes 22 and 26, and is detected by electrode 26.

Penis 14 increases in width and length during tumescence, or swelling, due to increased blood flow within penis 14. Nerves allow blood vessels at the base of penis 14 to increase in diameter to allow a greater rate of blood flow towards the penis. The greater amount of blood fills each of the sponge-like tissues of the first corpus cavernosum 30, second corpus cavernosum 32 and corpus spongiosum 34. As structures 30, 32 and 34 fill with blood, veins carrying blood away from penis 14 are collapsed due to the increasing size of the structures. The resulting filled structures 30, 32 and 34 change penis 14 from soft tissue into firm tissue that facilitates intercourse.

Accordingly, as the degree of penile tumescence increases, the distance D between electrodes 22, 26 increases. As the distance D increases, the impedance of penile tissue between electrodes 22, 26 increases. As discussed above, IMD 14 may sense the impedance, and thereby the degree of penile tumescence, based on the voltage and/or current of the electrical signal detected at electrode 26.

However, electrodes 22 and 26 do not need to be placed on the sides of penis 14 near the base to measure distance D. In other embodiments, the electrodes may be placed anywhere around the circumference of penis 14. For example, first electrode 22 may be placed underneath penis 14 while second electrode 26 may be placed on top of the penis. In addition, the exact diameter may not need to be detected. A line connecting electrodes 22 and 26 may not need to intersect with the center of penis 14. A slightly off-center measurement may still detect a change in tumescence. Electrodes 22, 26 that are placed approximately on opposite sides of penis 14 may be able to better sense tumescence then electrodes placed on the same side of penis.

Some embodiments may utilize the measurement of other dimensions of penis 14 to detect tumescence. For example, the length of penis 14 may indicate the level of tumescence. In this case, first electrode 22 may reside at the base of penis 14 while penile lead 24 travels the length of the penis to second electrode 26 at the distal tip. The impedance detected between both electrodes 22 and 26 would be representative of the length of penis 14 and corresponding tumescence level.

At least a portion of penile lead 24 that couples electrodes 22, 26 may be formed to allow expansion and contraction to comply with an extending and widening penis 14 during increasing tumescence. For example, penile lead may be at least partially constructed out of an elastic material. As another example, penile lead 24 may include a coiled, helical, or folded portion that is able to expand and contract during changing tumescence.

Further, the invention is not limited to embodiments that include a sensing lead 20 and penile lead 24 configured as shown in FIG. 2. In other embodiments, for example, each of electrodes 22, 26 may be coupled to IMD 16 by a respective sensing lead 20. As another example, sensing lead 20 may be bifurcated, and coupled each of electrodes 22, 26 to IMD 16.

In the illustrated example, electrodes 22, 26 are implanted just beneath the skin of penis 14 within the connective tissue surrounding first corpus cavemosum 30, second corpus cavemosum 32 and corpus spongiosum 34. Penile lead 24 may tunnel through the connective tissue following the circumference of penis 14 or the lead may be placed inside patient 12 to avoid changing tissue volume accompanying tumescence. While implanting electrodes 22 and 26 outside of structures 30, 32 and 34 may be less invasive to the erectile function, some embodiments may include placing electrodes 22 and 26 within first corpus cavemosum 30 and second corpus cavemosum 32, respectively.

Sensing lead 20, first electrode 22, penile lead 24 and second electrode 26 may be surgically or laparoscopically implanted within patient 12. Each component may be placed at one end and slowing pulled back to leave each component tunneled within tissue. Alternatively, each component may be separately implanted and subsequently connected by sensing lead 20 and penile lead 24.

Electrodes 22, 26 may be, for example, ring electrodes or pad electrodes. Further, electrodes 22, 26 may be formed as or to include fixation elements, such as hooks, barbs, helical structures, or tissue ingrowth mechanisms.

IMD 16 may be coupled to a stimulation lead 18 carrying one or more electrodes that are placed at a nerve site within the pelvic floor. For example, the electrodes may be positioned to stimulate the prostate parasympathetic nerve, the cavernous nerve, the pudendal nerve, or the sacral nerves to support and maintain an erection of penis 14. In particular, electrical stimulation may be applied to increase penile tumescence, i.e., blood flow into penis 14 that enables the patient to achieve an erection and participate in normal sexual activity. Further, the level of stimulation may be modified based on closed-loop feedback from electrodes 22 and 26 to maintain the tumescence of penis 14 at target level.

In this manner, IMD 16 may deliver stimulation therapy in order to achieve and maintain desired penile tumescence. At predetermined times, or at patient controlled instances, external programmer 28 may program IMD 16 to begin stimulation to achieve an erection. Upon the completion of sexual activity as indicated by a signal from external programmer 28, or after a predetermined period of time, IMD 16 may cease stimulation to allow the erection to subside.

During the course of stimulation, external programmer 28 or IMD 16 may adjust the stimulation delivered to the patient. For example, adjustment of stimulation parameters may be responsive to the electrical signal detected by IMD 16 at electrode 26. External programmer 28 or IMD 16 may adjust stimulation parameters, such as amplitude, pulse width, and pulse rate, based on the electrical signal. In this manner, external programmer 28 or IMD 16 adjusts stimulation to either increase or reduce penile tumescence based on the actual tumescence level detected of penis 14.

IMD 16 may sample tumescence information by emitting and detecting an electrical signal via electrodes 22, 26 periodically e.g., every few seconds, during the course of sexual activity. In other embodiments, programmer 28 may activate IMD 16, e.g., by wireless telemetry, to commence sensing. In some embodiments, IMD 16 or programmer 28 may alter tumescence sensing when there is an abrupt change in tumescence level, e.g., a tumescence level that exceeds a predetermined rate threshold, which indicates sexual arousal. In this case, IMD 16 may sense tumescence levels at relatively long intervals, and then sense at shorter intervals upon detection of the onset of sexual activity.

External programmer 28 may be a small, battery-powered, portable device that may accompany the patient 12 throughout the day or only during sexual activity. Programmer 28 may have a simple user interface, such as a button or keypad, and a display or lights. Patient 12 may initiate an erection, i.e., a voluntary increase in penile tumescence, via the user interface. In particular, in response to a command from the patient 12, programmer 28 may activate IMD 16 to deliver electrical stimulation therapy or alternatively deactivate IMD when no electrical stimulation therapy is desired. External programmer 28 may also receive input from patient 12 regarding the progress of therapy, which may be used by the programmer or IMD to control adjustment of therapy parameters. For example, patient 12 may signal that more or less tumescence is desired, or patient 12 may provide input via the external programmer that is relayed to IMD 16 relating to perceived pleasure or pain. In some embodiments, the length of time for an erection event may be determined by pressing a button a first time to initiate stimulation and a second time when the sexual activity is complete, or by a predetermined length of time permitted by programmer 28 or IMD 16. In each case, programmer 28 causes implantable IMD 16 to temporarily stimulate patient 12 to promote penile tumescence.

IMD 16 may be constructed with a biocompatible housing, such as titanium or stainless steel, and surgically implanted at a site in patient 12 near the pelvis. The implantation site may be a subcutaneous location in the side of the lower abdomen or the side of the lower back. One or more electrical stimulation leads 18 are connected to implantable IMD 16 and surgically or percutaneously tunneled to place one or more electrodes carried by the leads at a desired nerve site, such as a prostate parasympathetic, pudendal, sacral, or cavernous nerve site. In other embodiments, an IMD may be sized for implantation at the site to which stimulation or therapeutic substances are to be delivered, e.g., the pelvic floor. In such embodiments, the IMD need not be coupled to a lead or catheter, and may instead, as an example, deliver stimulation via electrodes formed on its housing.

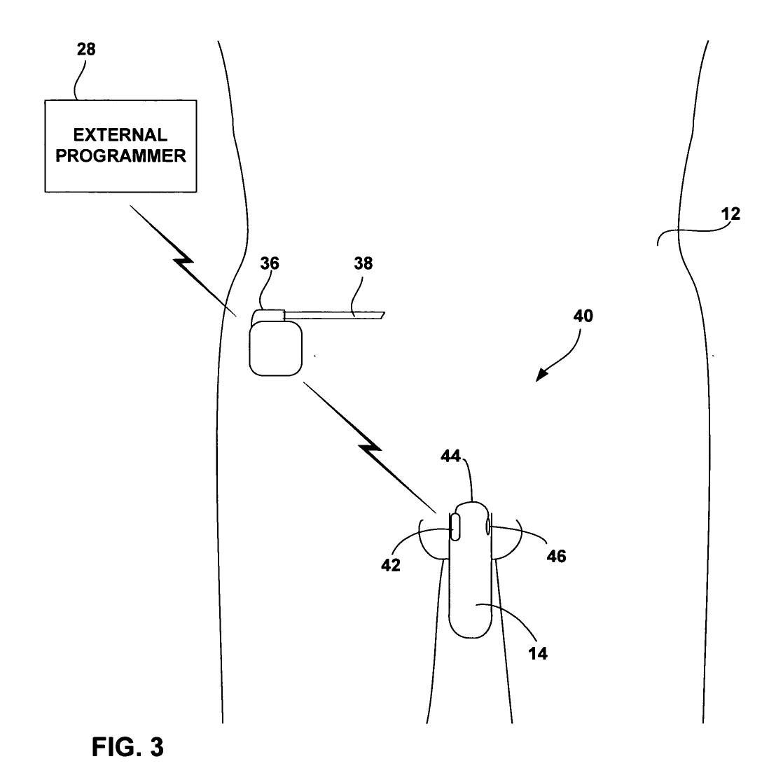

FIG. 3 is a schematic diagram illustrating another example system 40 that senses penile tumescence, the system including an implantable tumescence sensor 42. In the illustrated example, system 40 also includes an IMD 36 that delivers therapy to patient 12 for alleviation of sexual dysfunction, and an external programmer 28, as described above with reference to IMD 16 and FIGS. 1 and 2. In the illustrated example, IMD 36 delivers stimulation therapy via a lead 38, but may additionally or alternatively deliver one or more therapeutic substances via a catheter, as described above with reference to IMD 16 and FIGS. 1 and 2. Sensor 42 is separate from an IMD 36, and may be in wireless communication with one or both of IMD 36 and external programmer 28.

In the illustrated example, implantable sensor 42 is implanted within penis 14 and electrically coupled to an electrode 46 via a sensor lead 44. Sensor 42 emits an electrical signal via a housing electrode (not shown) included as part of the housing of sensor 42, and detects the electrical signal via electrode 46. In other embodiments, sensor 42 emits the electrical signal via electrode 46, and detects the signal via the housing electrode.

As discussed above, the detected electrical signal is indicative of the degree of tumescence of penis 14. Sensor 42 may wirelessly transmit information relating to the detected electrical signal to one or both of IMD 36 and external programmer 28, which may control delivery of therapy by IMD 36 based on the signal. As examples, sensor 42 may transmit one or both of the voltage or current amplitude of the detected signal, or the impedance of penile tissue determined based on the voltage or current, to the programmer or IMD. IMD 36 may deliver therapy based on the current, voltage, impedance, changes therein, or programmer 28 may control delivery of therapy by IMD 36 through commands wirelessly transmitted to IMD 36 based on the received current, voltage or impedance, or changes therein, as discussed above with reference to FIGS. 1 and 2. In this manner, the information transmitted from sensor 42 to one or both of programmer 28 or IMD 36 may facilitate closed loop feedback of erectile state information during the course of sexual activity.

Additionally, one or more of sensor 42, IMD 36 and external programmer 28 may store the sensed voltages, currents or impedances, and thereby store indications of penile tumescence over time for short or long-term monitoring of penile tumescence. In either case, such penile tumescence information may be presented to a user, such as a physician, via external programmer 28, or another computing device. Further, the invention is not limited to embodiments that include a therapy delivering IMD 36. In some embodiments, sensor 42 and/or external programmer 28 may store sensed penile tumescence information without delivery of therapy, for short or long-term monitoring of penile tumescence.

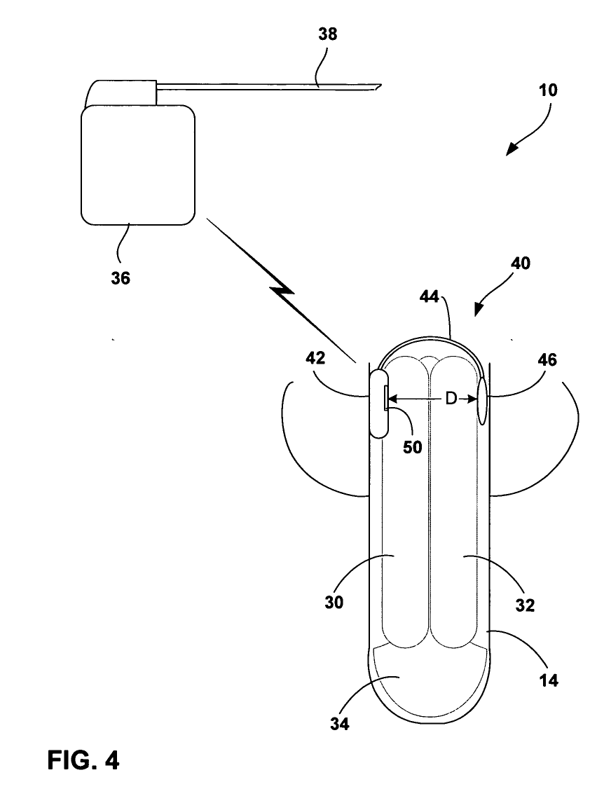

FIG. 4 is a schematic diagram further illustrating system 40. As shown in FIG. 4, implantable sensor 42 includes a housing electrode 50 formed on the housing of sensor 42, and sensor 42 is electrically coupled to another electrode 46 by a sensor lead 44. Electrodes 46, 50 are implanted at first and second locations within penis 14. More particularly, in the illustrated example, electrode 50 is formed on the housing of sensor 42, which is located near the base of the shaft of penis 14 on a first side. Electrode 46 is disposed near the base of the shaft of penis 14 on a second side, e.g., approximately opposite electrode 50. An electrical signal emitted by one of electrodes 46, 50 travels approximately the distance D between electrodes 46, 50, and is detected by the other of electrodes 46, 50.

As discussed above with reference to FIG. 2, as the degree of penile tumescence increases, the distance D increases. As the distance D increases, the impedance of penile tissue between electrodes 46, 50 increases. Sensor 42 senses the impedance, and thereby the degree of penile tumescence, based on the voltage and/or current of the electrical signal detected at one of electrodes 46, 50.

However, sensor 42 (including housing electrode 50) and electrode 46 do not need to be placed on the sides of penis 14 near the base to measure distance D. In other embodiments, sensor 42 and electrode 46 may be placed anywhere around the circumference of penis 14. For example, sensor 42 may be placed underneath penis 14 while sensor electrode 46 may be placed on top of the penis. In addition, the exact diameter may not need to be detected. A line connecting electrodes 50 and 46 may not need to intersect with the center of penis 14. A slightly off-center measurement may still detect a change in tumescence. In general, electrodes 46, 50 placed approximately on opposite sides of penis may be better able to sense tumescence than electrodes placed on the same side of penis.

Some embodiments may utilize the measurement of other dimensions of penis 14 to detect tumescence. For example, the length of penis 14 may indicate the level of tumescence. In this case, sensor 42 may reside at the base of penis 14 while sensor lead 44 travels the length of the penis to electrode 46 at the distal tip. The impedance detected between electrodes 46, 50 would be representative of the length of penis 14 and corresponding tumescence level.

At least a portion of sensor lead 44 may be formed to allow expansion and contraction to comply with an extending and widening penis during increasing tumescence. For example, sensor lead 44 may be at least partially constructed out of an elastic material. As another example, sensor lead 44 may include a coiled, helical, or folded portion that is able to expand and contract during changing tumescence.

In the illustrated example, sensor 42 and electrode 46 are implanted just beneath the skin of penis 14 within the connective tissue surrounding first corpus cavernosum 30, second corpus cavernosum 32 and corpus spongiosum 34. Sensor lead 44 may tunnel through the connective tissue following the circumference of penis 14 or the lead may be placed inside patient 12 to avoid changing tissue volume accompanying tumescence. While implanting sensor 42 and sensing electrode 46 outside of structures 30, 32 and 34 may be less invasive to the erectile function, some embodiments may include placing sensor 42 and sensor electrode 46 within first corpus cavernosum 30 and second corpus cavemosum 32.

Implantable sensor 42, sensor lead 44 and sensor electrode 46 may be surgically or laparoscopically implanted within patient 12. Each component may be placed at one end and slowing pulled back to leave each component tunneled within tissue. Alternatively, sensor 42 and sensor electrode 46 may be separately implanted and subsequently connected by sensor lead 44.

Electrodes 46, 50 may be, for example, ring electrodes or pad electrodes. Further, electrodes 46, 50 may be formed as or to include fixation elements, such as hooks, barbs, helical structures, or tissue ingrowth mechanisms. Additionally or alternatively, the housing of sensor 42 may be formed to include or coupled to one or more fixation elements, such as hooks, barbs, helical structures, or tissue ingrowth mechanisms. As another example, the housing of sensor 42 may include or be coupled to a hydrogel material that expands when exposed to fluid to substantially fix sensor 42 at the location at which it is implanted.

Programmer 28 or IMD 36 may activate sensor 42, e.g., by wireless telemetry, to commence sensing. Sensor 42 may perform each tumescence measurement in response to a request from IMD 36 or programmer 28. Alternatively, sensor 42 may detect the tumescence level periodically e.g., every few seconds, during the course of sexual activity. In some embodiments, sensor 42 may make tumescence measurements when there is an abrupt change in tumescence level, e.g., a tumescence level that exceeds a predetermined rate threshold, which indicates sexual arousal. In this case, sensor 42 may sense tumescence levels at relatively long intervals, and then sense at shorter intervals upon detection of the onset of sexual activity.

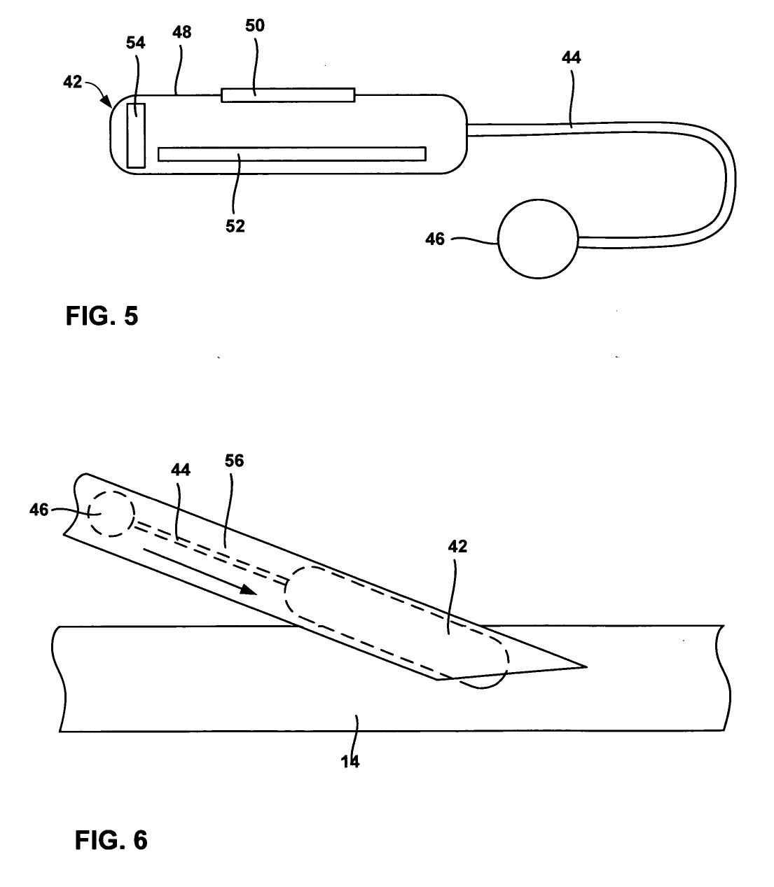

FIG. 5 is an enlarged, cross-sectional side view of sensor 42, lead 44 and electrode 46 of FIGS. 3 and 4, according to an example configuration. Implantable sensor 42 includes sensor housing 48, housing electrode 50, circuit board 52 and power supply 54. Electrode 46 is electrically coupled to sensor 42 via sensor lead 44. Sensor housing 48 of implantable sensor 42 is embedded in the connective tissue of penis 14. In the illustrated example, sensor housing 48 is in the shape of a rounded capsule and includes a smooth surface. The invention is not limited to this example configuration.

Housing electrode 50 and electrode 46 are coupled to circuit board 52 within implantable sensor 42. A power source 54, such as a battery, provides to power circuit board 52, housing electrode 50 and sensor electrode 46. Circuit board 52 includes processing electronics, and signal generation and sensing electronics for emitting and detecting the electrical signal via electrodes 46 and 50. In addition, circuit board 52 may include telemetry circuitry for wireless communication with IMD 36, external programmer 28, or both.

Power source 54 may take the form of a small rechargeable or non-rechargeable battery, which may be configured as a coin cell or pin cell. Different types of batteries or different battery sizes may be used, depending on the requirements of a given application. To promote longevity, power source 54 may be rechargeable via induction or ultrasonic energy transmission, and includes an appropriate circuit for recovering transcutaneously received energy. For example, power source 54 may include a secondary coil and a rectifier circuit for inductive energy transfer. Power generation or charging electronics may be carried on circuit board 52. In still other embodiments, power source 54 may not include any storage element, and sensor 42 may be fully powered via transcutaneous inductive energy transfer.

As a further alternative, IMD 36 or programmer 28 may be configured to apply inductive power to sensor 42 whenever detection is desired. In this case, when inductive power is not applied, sensor 42 is asleep. Upon application of inductive power, sensor 42 wakes up, acquires a sense signal, and transmits the signal to programmer 28 or IMD 36. Accordingly, in such embodiments, IMD 36 or programmer 28 determines the sampling rate of sensor 42 by powering up the sensor at desired intervals.

Sensor 42 may rest in a cavity formed within the connective tissue near the surface of penis 14. Sensor 42 may have a capsule-like shape, and may have, as examples, a length of approximately 2 to 10 mm, a width of approximately 2 to 5 mm, and a thickness of approximately 1 to 5 mm. The capsule-like shape may produce a circular cross-section, in which case sensor 42 may have a diameter of approximately 1 to 5 mm, rather than width and height dimensions. In some embodiments, housing 48 may be of a more compatible shape the anatomy of the implant site. For example, sensor 42 may be placed within tissue that includes a curved surface. A capsule-like housing 48 may cause the housing to protrude away from penis 14. A concave shape of housing 48 may follow the contours of the curved side of penis 14 and make the sensor 42 less detectable beneath the skin of penis 14. Housing 48 may be formed of one or more biocompatible materials, such as titanium, stainless steel, epoxy, or polyvinylchloride.

FIG. 6 is a schematic diagram illustrating implantation of sensor 42 within the penis. Sensor 42 may be implanted using minimally invasive techniques. For example, a surgeon may inject sensor 42, lead 44 and electrode 46 into the connective tissue of penis 14 using a needle 56, as shown in FIG. 6. Needle 56 is constructed of a metal alloy and comprises a hollow cylinder and a pointed distal end for puncturing the skin of penis 14. Needle 56 includes sensor 42, and may include a fluid to force the sensor out of the needle. An exemplary fluid may be saline or other biocompatible fluid. In other embodiments, needle 56 may comprise a catheter or other hollow delivery vehicle.

Once needle 56 in positioned at the appropriate location of penis 14, the surgeon may force sensor 42 into place. Removing needle 56 from penis 14 allows the connective tissue close and surround, or partially surround, sensor 42, lead 44, and electrode 46. In some embodiments, the surgeon may suture the insertion hole to promote tissue healing. The suture may comprise resorbable or non-resorbable suture or staples. Unnecessary openings within corpus cavemosum 30 or 32 may be avoided to prevent blood loss during tumescence events, infection or other health problems. In other embodiments, sensor 42 may be implanted through more invasive procedures, such as open cutting open the skin of penis 14 and suturing the entire implantation site.

As discussed above, in some embodiments, implantable sensor 42 may carry or include one or more fixation elements that help to anchor the sensor within the connective tissue of penis 14. Such fixation elements may include hooks, barbs, helical elements, tissue ingrowth mechanisms, or hydrogel elements. For embodiments that include hydrogel elements, during implantation, the hydrogel elements are in a dehydrated state, in which the hydrogel elements are smaller. But when implanted in the body of a patient, the hydrogel elements absorb water from the body tissues and assume a larger, hydrated state. One or more expanded hydrogel elements may resist migration of the sensor 42 within penis 14.

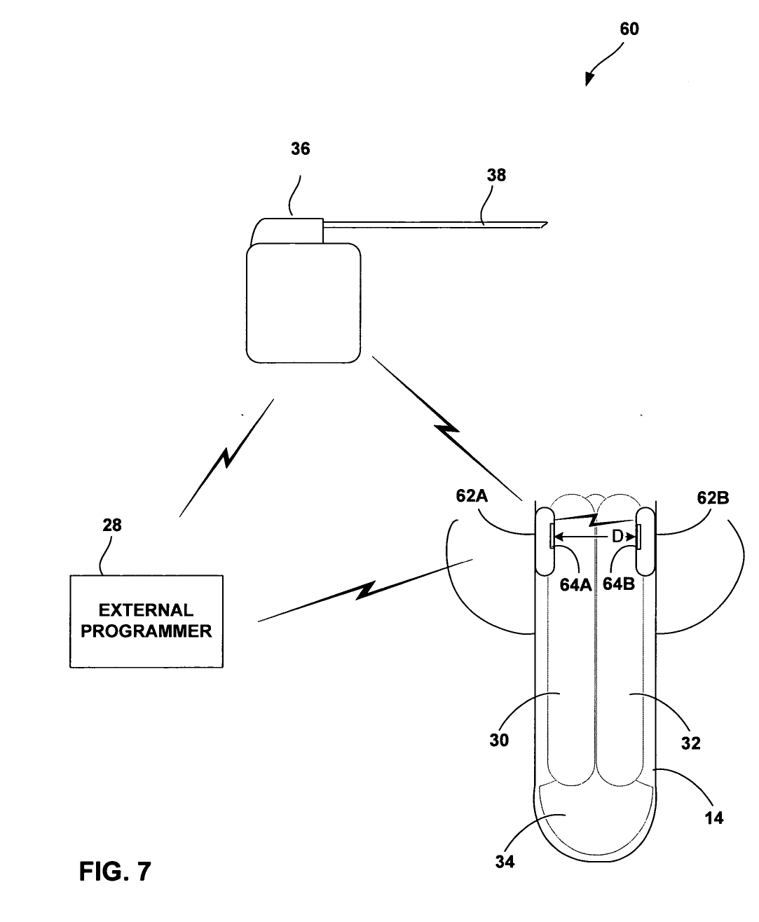

FIG. 7 is a schematic diagram illustrating another example system 60 that senses penile tumescence, the system including two separate implantable modules 62A and 62B (collectively, “modules 62”). In the illustrated example, each of modules 62 includes a respective one of housing electrodes 64A and 64B (collectively, “electrodes 64”). In other embodiments, rather than housing electrodes 64, one or both of modules 62 may be electrically coupled to an electrode via a lead. Because system 60 includes two separate modules 62 with respective electrodes 64, it may not need to include a lead 44 tunneled a significant distance through tissues of penis 14. Although illustrated as implanted on opposite sides of the base of penis 14, modules 62 may be implanted at different locations anywhere within and along penis 14, as described above with reference to FIGS. 1-6.

Modules 62 cooperate to enable sensing of penile tumescence. One of modules 62 emits an electrical signal via its respective electrode, while the other module detects the signal via its respective electrode. The detecting module 62 may transmit information relating to the detected signal, which is indicative of the degree of penile tumescence, to one or both of IMD 36 or external programmer 28, as described above with respect to sensor 42. The activities of modules 62, e.g., emission and detection of the electrical signal, may be controlled by one or both of IMD 36 and programmer 28 via wireless communication, in the manner described above with respect to sensor 42.

Further, as illustrated in FIG. 7, modules 62 may wirelessly communicate with each other. For example, one of modules 62 may act as an intermediary for communication between the other module and one or both of IMD 36 and external programmer 28. Further, modules 62 may communicate to coordinate emission and detection of the electrical signal. In embodiments in which the impedance of tissue between electrodes 64 is actually determined, one of the modules 62 may communicate the voltage and/or current of the emitted or detected signal to the other of the modules, so that the other of the modules is able to determine the impedance. In other embodiments, the modules may communicate such information to IMD 36 or programmer 28 for determination of the impedance. In still other embodiments, the impedance is not determined. In some embodiments, the detecting module 62 may merely communicate a voltage and/or current of the detected signal to IMD 36 or programmer 28, which may control delivery of therapy based on the voltage and/or current.

Each of modules 62 may be constructed substantially similarly to module 42 as illustrated in FIG. 5. Further, each of modules may be implanted using the techniques depicted and described above with respect to module 42 and FIG. 6. Each of modules 62 may include or be coupled to one or more fixation elements, as described above with respect to module 42.

Additionally, one or more of modules 62, IMD 36 and external programmer 28 may store the voltages, currents or impedances, sensed or determined by modules 62, and thereby store indications of penile tumescence over time for short or long-term monitoring of penile tumescence. In either case, such penile tumescence information may be presented to a user, such as a physician, via external programmer 28, or another computing device. Further, the invention is not limited to embodiments that include a therapy delivering IMD 36. In some embodiments, modules and/or external programmer 28 may store sensed penile tumescence information without delivery of therapy, for short or long-term monitoring of penile tumescence.

While FIGS. 2 and 4 provide examples of possible detection devices for detecting tumescence of penis 14, other detectors or sensors may be used. Any sensor may be used which includes the ability to sense a change in tumescence level, transmit information to other devices, and operate within patient 12.

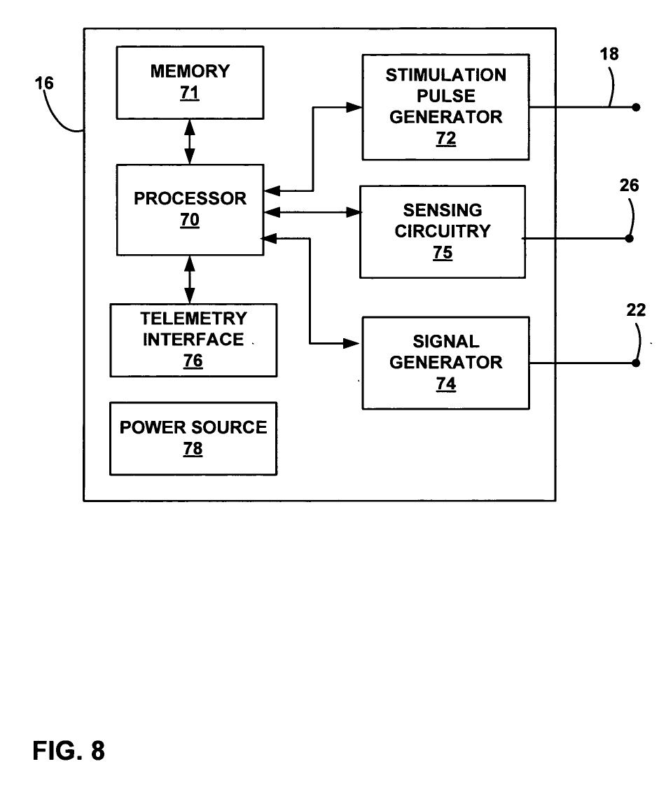

FIG. 8 is a functional block diagram illustrating various components of implantable IMD 16 of FIGS. 1 and 2 according to an example embodiment. In the example of FIG. 7, IMD 16 includes a processor 70, memory 71, stimulation pulse generator 72, signal generation circuitry 74, sensing circuitry 75, telemetry interface 76, and power source 78. Memory 71 may store programs instructions for execution by processor 70, which, when executed by processor 70, cause IMD 16 and processor 70 to perform the function ascribed to them herein. Memory 71 may also store stimulation therapy data, e.g., therapy parameters such as pulse amplitude, rate and width. Memory 71 may also store look-up tables, functions, thresholds, or the like, which processor 70 may use to control therapy based on tumescence information, e.g., based on a voltage, current or impedance related to an electrical signal sensed via electrode 26 and sensing circuitry 75. Memory 71 may also store such tumescence information for long or short-term monitoring of penile tumescence. Memory 71 may include any one or more of RAM, ROM, EEPROM, flash memory or the like. Processor 70 may include any one or more of a microprocessor, DSP, ASIC, FPGA, or other digital logic circuitry.

Processor 70 controls signal generation circuitry 74, which may include current generation known in the art, to emit an electrical signal via electrode 22. Processor 70 detects the electrical signal via electrode 26 and sensing circuitry 75, which may include voltage or current measuring circuits known in the art. Processor 70 may trigger measurements, e.g., emission and detection of the signal, on a continuous basis, at periodic intervals, or upon request from external programmer 28. Alternatively, or additionally, processor 70 may increase the monitoring of tumescence information when there is an abrupt change in the tumescence level, e.g., at the onset of sexual arousal.

Processor 70 controls stimulation pulse generator 72 to deliver electrical stimulation therapy via one or more leads 18 based on the signal detected via electrode 26 and sensing circuitry 75. For example, processor 70 may determine whether to initiate, terminate or adjust therapy based on a voltage, current and/or impedance associated with the signal. Processor 70 may compare such information to one or more thresholds, look-up tables, or the like, and determine whether to initiate, terminate or modify delivery of therapy based on the comparison. In this manner, processor 70 may directly control therapy in response to information received from sensing circuitry 74. Alternatively, programmer 28 may receive tumescence information from processor 70 via telemetry interface 76, and provide commands controlling therapy parameter adjustments to processor 70 via the telemetry interface.

As an example, if the tumescence information indicates an inadequate tumescence level during a desired erectile event, processor 70 may increase the amplitude, pulse width or pulse rate of the electrical stimulation applied by stimulation pulse generator 72, or change electrode combination or polarity, to increase stimulation intensity, and thereby increase penile tumescence. If tumescence is adequate, processor 70 may implement a cycle of downward adjustments in stimulation intensity until the tumescence level becomes inadequate, and then incrementally increase the stimulation upward until tumescence is again adequate. In this way, processor 70 converges toward an optimum level of stimulation. Although processor 70 is generally described in this example as adjusting stimulation parameters, it is noted that the adjustments may be generated by external programmer 28, as mentioned above.

In some embodiments, IMD 16 may additionally provide an evaluation algorithm in which processor 70 sequentially adjusts the therapy parameters, e.g., according to a lookup table or set of equations stored within memory 71, to identify a parameter combination that is “best” in terms of tumescence or other factors. For example, processor 70 may systematically try to find the set of amplitude, frequency, pulse width and waveform that provides the greatest tumescence for patient 12, as indicated by the voltage, current or impedance associated with the signal detected by the electrodes implanted within the patient's penis. Once the best set of parameters has been discovered, processor 70 may store the parameters for use and exit the evaluation algorithm. The evaluation algorithm may be revisited at any time as requested by patient 12, a physician, or processor 70.

During adjustment of stimulation parameters based on tumescence information, e.g., during feedback operation, or execution of an evaluation algorithm, patient 12 may provide real-time feedback via programmer 28. During execution of the evaluation algorithm, such feedback may be used with tumescence information to score a particular parameter set. Such feedback may indicate, as examples, the degree of sensation or pleasure, or the degree of discomfort or pain, experienced by patient 12 during stimulation with a particular parameter set. During feedback operation, processor 70 may adjust therapy based on tumescence information, as described above, and also based upon such patient feedback. For example, patient 12 may provide feedback relating to the degree of sensation or pleasure, and processor 70 may adjust therapy based on the tumescence information and the indicated degree of sensation or pleasure. Further, if patient 12 experiences discomfort or pain during delivery of, patient 12 may use programmer 28 to indicate the degree of pain, which processor 70 may consider with tumescence information and, in some embodiments, degree of sensation, to control delivery of therapy, e.g., adjustment of parameters.

During feedback operation or execution of an evaluation program, patient 12 may use programmer 28 to direct processor 70 to instantly stop all stimulation, e.g., based on pain experienced by patient. The therapy parameter values currently active when such an event occurs may be stored as “blacklisted” values, e.g., to be avoided, or threshold values which should not be traversed during adjustment of the therapy parameters. As an additional safety mechanism, processor 70 or programmer 28 may compare the current stimulation time to a maximum therapy duration as predetermined by the physician or patient 12. Processor 70 may stop stimulation if therapy has continued for a duration longer than allowed.

As discussed above, in some embodiments, IMD 16 may deliver stimulation pulses with different parameters for different phases of sexual activity, such as arousal and ejaculation. For a first phase of arousal, IMD 16 may deliver stimulation pulses at a frequency in the range of approximately 50 to 150 Hz, and more preferably approximately 70 to 100 Hz. Each pulse for the first phase may have an amplitude in the range of approximately 1 to 10 volts, and more preferably approximately 2 to 5 volts, and a pulse width in the range of approximately 100 to 400 microseconds, and more preferably approximately 200 to 300 microseconds. The duration of the first phase of stimulation may depend on a detected transition to the second phase, which may be indicated by sensed tumescence.

For a second phase of ejaculation, IMD 16 may deliver stimulation pulses at a frequency in the range of approximately 1 to 5 Hz, or in the range of approximately 25 to 35 Hz. Each pulse for the second phase may have an amplitude in the range of approximately 1 to 10 volts, and more preferably approximately 2 to 5 volts, and a pulse width in the range of approximately 200 to 700 microseconds, and more preferably approximately 400 to 500 microseconds.

In some embodiments, processor 70 may disregard a signal received from sensing circuitry 74 if an unusual electrical environment is detected within penis 14. For example, if a signal representative of urination is detected, processor 70 may delay sensing the tumescence of penis 14 for a fixed period of time or until urination has ceased. In this case, processor 70 may suspend tumescence sensing until the signal detected by sensing circuitry 74 is within an operable range.

Penis size may change due to a variety of factors, such as an activity type or activity level of patient 12. Hence, for a given set of stimulation parameters, the efficacy of stimulation may vary in terms of rate of tumescence increase or decrease, due to changes in the physiological condition of the patient. For this reason, the continuous or periodic availability of tumescence information from sensing circuitry 74 is highly desirable.

With this tumescence information, IMD 16 is able to respond to changes in penis 14 size with dynamic adjustments in the stimulation parameters delivered to patient 12. In particular, processor 70 is able to adjust parameters in order to improve blood flow to penis 14 or decrease blood flow away from the penis. In some cases, the adjustment may be nearly instantaneous.

In general, if penis 14 is decreasing in tumescence for an unknown reason, processor 70 may dynamically increase the level of therapy to be delivered to stop or reverse the decreasing tumescence. Conversely, if penis 14 is increasing in tumescence consistently, processor 70 may incrementally reduce stimulation, e.g., to conserve power resources, until the tumescence level reaches a threshold upper limit. Increases or reductions in the level of therapy may include upward or downward adjustments in amplitude (current or voltage), pulse width, or pulse rate of stimulation pulses delivered to patient 12.

Telemetry interface 76 may include at least one antenna and circuitry for radio frequency (RF) communication or proximal inductive interaction of IMD 16 with external programmer 28. Power source 78 of IMD 16 may be constructed somewhat similarly to power source 54. For example, power source 68 may be a rechargeable or non-rechargeable battery, or alternatively take the form of a transcutaneous inductive power interface.

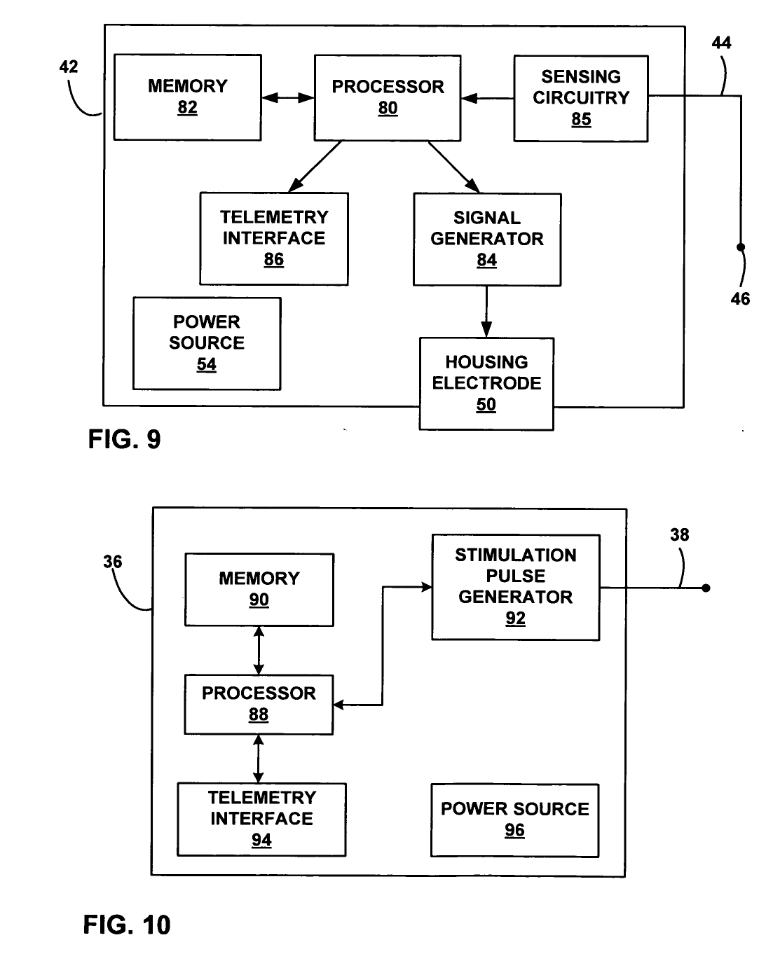

FIG. 9 is functional block diagram illustrating various components of an exemplary wireless implantable tumescence sensor 42. In the example of FIG. 9, implantable tumescence sensor 42 includes a processor 80, memory 82, signal generation circuitry 84, sensing circuitry 85, telemetry interface 86, power source 54 and housing electrode 50. Sensor lead 44 connects sensor electrode 46 to sensing circuitry 85. Signal generation and sensing circuitry 84, 85 may be carried on a circuit board 52, along with processor 80, memory 82 and telemetry interface 86. Housing electrode 50 transmits electrical signals across penis 14 for detection by sensor electrode 46, the detected signal representative of penis size or tumescence. Signal generation circuitry 74 may include current generators known in the art to generate such signals. The electrical signals may be amplified, filtered, and otherwise processed as appropriate by sensing circuitry 85, which may include circuitry for measuring voltage or current. In some embodiments, the signals may be converted to digital values and processed by processor 80 before being saved to memory 82 or sent to implantable IMD 36 as tumescence information via telemetry interface 86.

Memory 82 stores program instructions for execution by processor 80 and tumescence information generated by sensing circuitry 85. Tumescence information may be sent to implantable IMD 36 or external programmer 28 for presentation to a user or use to control delivery of therapy to patient 12. Memory 82 may include any one or more of RAM, ROM, EEPROM, flash memory or the like. Processor 80 may include any one or more of a microprocessor, DSP, ASIC, FPGA, or other digital logic circuitry.

Processor 80 may control telemetry interface 86 to send tumescence information to IMD 36 or programmer 28 on a continuous basis, at periodic intervals, or upon request from IMD 36 or programmer 28. Telemetry interface 86 may include one or more antennae and circuitry for radio frequency (RF) communication or proximal inductive interaction of sensor 42 with programmer 28.

Power source 54 delivers operating power to the components of implantable sensor 42. As mentioned previously, power source 54 may include a small rechargeable or non-rechargeable battery and a power generation circuit to produce the operating power. Recharging may be accomplished through proximal inductive interaction between an external charger and an inductive charging coil within sensor 42. In some embodiments, power requirements may be small enough to allow sensor 42 to utilize patient motion and implement a kinetic energy-scavenging device to trickle charge a rechargeable battery. In other embodiments, traditional batteries may be used for a limited period of time. As a further alternative, an external inductive power supply could transcutaneously power sensor 42 whenever tumescence measurements are needed or desired.

In some embodiments, sensor 42 may be deployed purely as a diagnostic device to obtain and store penile tumescence measurements over a period of time. In particular, sensor 42 may be used to diagnose a patient's condition in order to determine whether the patient suffers from erectile dysfunction, the degree the dysfunction, and whether electrical stimulation therapy may be effective. In each case, sensor 42 is entirely ambulatory and requires little or no setup by the patient 12. Instead, sensor 42 simply accompanies patient 12 throughout his daily routine. Loop recorder functionality may be especially desirable for monitoring of penile tumescence over an extended period of time. Following implantation of IMD 36, sensor 42 may function as both a diagnostic device and a closed loop feedback device for the IMD.

Modules 62 of FIG. 7 may be include components substantially similar to those of sensor 42 illustrated in FIG. 9. However, each of modules 62 may include a housing electrode, rather than a lead 44 and electrode 46, and may include only one of a signal generator and sensing circuitry.

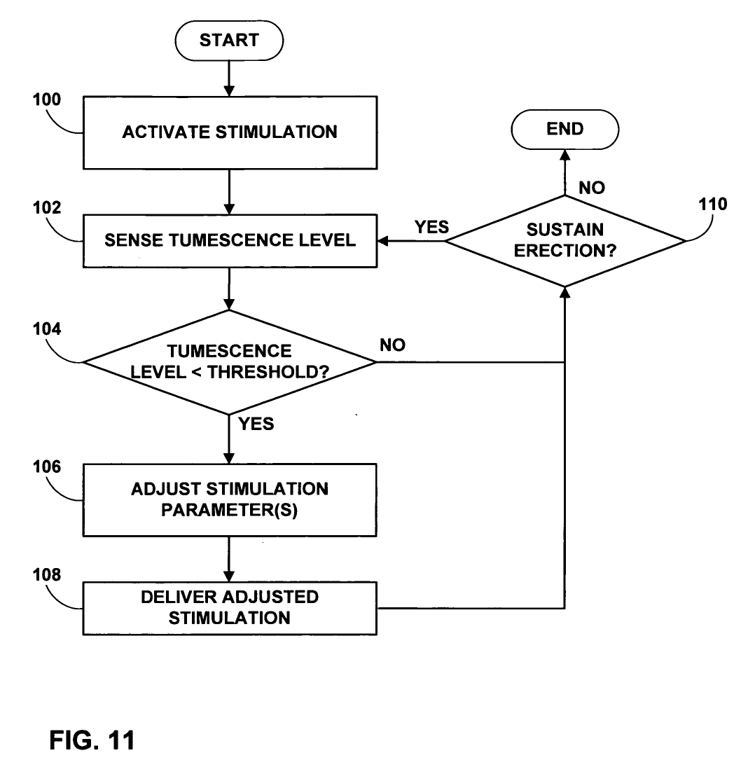

FIG. 10 is a functional block diagram illustrating various components of an exemplary implantable IMD 36 for use with implantable sensor 42. With the exception of not containing sensing circuitry, IMD 36 is very similar to IMD 16 of FIG. 8. In the example of FIG. 10, IMD 36 includes a processor 88, memory 90, stimulation pulse generator 92, telemetry interface 94 and power source 96, which are substantially similar to the corresponding components of IMD 16 discussed above with reference to FIG. 8. Processor 88 receives tumescence information, e.g., voltages, currents and/or impedances, from sensor 42 via telemetry interface 94. Based on such information, processor 88 may determine whether therapy should be initiated, terminated, or adjusted, as described above. Processor 88 may receive such information from sensor 42 on a continuous basis, at periodic intervals, or in response to a request made by processor 88 via telemetry interface 94. Alternatively, or additionally, processor 88 may direct sensor 42 to increase the monitoring of tumescence information when there is an abrupt change in the tumescence level, e.g., at the onset of sexual arousal. Processor 88 may control therapy based on tumescence information independently, or, where external programmer 28 receives tumescence information from sensor 42, in response to programming changes from external programmer 28. Processor 88 may provide feedback control of therapy parameters and a therapy parameter evaluation program, as described above with reference to processor 70. Processor 88 may receive feedback from patient 12 via external programmer 28, and use such feedback during closed-loop feedback operation and/or the evaluation program, as described above with reference to processor 70.

Telemetry interface 94 may include antennae and circuitry for radio frequency (RF) communication or proximal inductive interaction with implantable sensor 42 and/or external programmer 28. Also, power source 96 of IMD 36 may be constructed somewhat similarly to power source 54. For example, power source 96 may be a rechargeable or non-rechargeable battery, or alternatively take the form of a transcutaneous inductive power interface.

FIG. 11 is a flow chart illustrating a technique for delivery of stimulation therapy to alleviate sexual dysfunction based on closed loop feedback from an implantable tumescence sensor. In the example of FIG. 11, IMD 16 makes use of information obtained from electrodes 22 and 26 and external programmer 28. Implantable IMD 36 and sensor 42 or modules 62 may be utilized in this technique as well.

A patient 12 activates stimulator 16 (100) by entering a command via a user interface associated with external programmer 28. The command indicates that the patient would like to commence sexual activity. In response to the command, programmer 28 activates IMD 16 (90) to deliver stimulation therapy.

During the course of stimulation therapy, electrodes 22 and 26 are utilized by IMD 16 to sense the degree of tumescence of penis 14 (102) e.g., based on one or more of voltage, current or impedance associated with a signal detected via one of electrodes 22, 26. If IMD 16 or, in some embodiments, programmer 28 determines that the tumescence is below an applicable threshold (104), indicating an inadequate erectile state, one or more stimulation parameters may be adjusted (106) to provide therapy that increases tumescence. The adjustment may be made directly by IMD 16, or by IMD 16 in response to a command received from programmer 28.

Upon delivery of the adjusted stimulation (108), IMD 16 or programmer 28 determines whether the patient 12 wants to sustain the erection (110), or whether sexual activity has terminated. Patient 12 may terminate sexual activity by entry of a command via a user interface associated with programmer 28. If sustained erection is desired, the process continues with tumescence sensing (102), threshold comparison (104), adjustment of stimulation parameters (106) and delivery of adjusted stimulation (108).

In other embodiments, IMD 16 may continuously cycle stimulation to conserve power. If the tumescence level is greater than the threshold (104), IMD 16 may slightly decrease stimulation before determining if patient 12 desires to continue the erection (110). Adding this step in the processes may help to decrease the operating power required to stimulate patient 12.

Further, as described above, IMD 16, 36 may additionally provide an evaluation algorithm in which the IMD sequentially adjusts the therapy parameters, e.g., according to a lookup table or set of equations stored within a memory, to identify a parameter combination that is “best” in terms of tumescence or other factors. For example, the IMD may systematically try to find the set of amplitude, frequency, pulse width and waveform that provides the greatest tumescence for patient 12, as indicated by the voltage, current or impedance associated with the signal detected by the electrodes implanted within the patient's penis. Once the best set of parameters has been discovered, the IMD may store the parameters for use and exit the evaluation algorithm. In some embodiments, an external programmer 28 may direct the IMD deliver therapy according to a variety of parameters, and may itself evaluate the therapy parameters. The evaluation algorithm may be performed initially in a clinic shortly after implantation of a system as described herein, and revisited at any time as requested by patient 12, a physician, the IMD, or the external programmer.

In some embodiments, as mentioned previously, implantable sensor 42 or modules 62 may be used exclusively for monitoring tumescence without providing feedback for stimulation therapy. In this case, sensor 42 or modules 62 simply collect data and either store it locally, or send it to external programmer 28 for presentation of tumescence information to a user, such as a physician. Tumescence may be measured continuously, intermittently or at the request of external programmer 28. These embodiments may be used for, as examples, disease diagnosis or condition monitoring, and may allow a patient to avoid frequent clinic visits and uncomfortable procedures while acquiring more extensive and more accurate tumescence data during sexual activity.

Although the invention has been generally described in conjunction with implantable stimulation devices, tumescence sensing, sensors 42 and/or modules 62 may also be used with other implantable medical devices, such as implantable drug delivery devices, which may be configured to treat sexual dysfunction. In particular, tumescence levels may be used to trigger and control delivery of any of a variety of drugs capable of achieving arousal in a male or female patient from a chemical stimulation device. Prostaglandin, Alprostdil, Tadalafil, Sildenafil, Vardenfil are examples of drugs that could be infused, e.g., by intracavernous injection, to elicit an erection in a male patient. Approximate dosages for some of the above drugs are: Alprostdil—10 to 250 micrograms, Sildenafil—10 to 250 micrograms, and Apormorphine—10 to 250 micrograms. The tumescence levels obtained by sensor 12 may be used to trigger drug delivery, control the rate of delivery of the drug, or control the overall amount of drug delivered to the patient, e.g., to achieve and maintain an erection during a first phase of sexual activity. A suitable drug delivery system is described in the aforementioned pending application to Gerber.

Various embodiments of the described invention may include processors that are realized by microprocessors, Application-Specific Integrated Circuits (ASICs), Field-Programmable Gate Arrays (FPGAs), or other equivalent integrated or discrete logic circuitry. The processor may also utilize several different types of storage methods to hold computer-readable instructions for the device operation and data storage. These memory or storage media may include random access memory (RAM), electronically-erasable programmable read only memory (EEPROM), or flash memory, e.g. Compact Flash or Smart Media. Each storage option may be chosen depending on the embodiment of the invention

Many embodiments of the invention have been described. Various modifications may be made to the described embodiments without departing from the scope of the claims. These and other embodiments are within the scope of the following claims.

1. A method comprising:
emitting an electrical signal from a first electrode implanted within a penis of a patient at a first location;
detecting the electrical signal via a second electrode implanted within the penis at a second location; and
delivering therapy from an implantable medical device to the patient based on the detected electrical signal to control penile tumescence.

2. The method of claim 1, wherein delivering therapy to control penile tumescence comprises delivering therapy to at least one of initiate or sustain an erection.

3. The method of claim 1, wherein delivering therapy comprises delivering electrical stimulation.

4. The method of claim 3, wherein delivering electrical stimulation comprises delivering electrical stimulation to at least one of a prostate parasympathetic nerve, a cavernous nerve, pudendal nerve, or a sacral nerve.

5. The method of claim 1, wherein delivering therapy based on the detected electrical signal comprises delivering therapy based on at least one of a voltage or a current of the detected electrical signal.

6. The method of claim 5, wherein delivering therapy based on at least on of a voltage or a current comprises:
determining an impedance based on at least one of the voltage or the current; and
delivering therapy based on the impedance.

7. The method of claim 1, wherein at least the second electrode is electrically coupled to the implantable medical device, and detecting the electrical signal via a second electrode comprises detecting the electrical signal at the implantable medical device.

8. The method of claim 1, wherein at least the second electrode is electrically coupled to an implantable sensor that is separate from the implantable medical device, and detecting the electrical signal comprises detecting the electrical signal at the implantable sensor.

9. The method of claim 8, further comprising transmitting information relating to the detected electrical signal from the implantable sensor to an external programmer.

10. The method of claim 9, further comprising transmitting signals from the external programmer to the implantable medical device based on the information to control the delivery of therapy from the implantable medical device to the patient.

11. The method of claim 9, further comprising presenting penile tumescence information to a user via the external programmer based on the information received from the implanted sensor.

12. The method of claim 8, further comprising transmitting information relating to the detected electrical signal from the implantable sensor to the implantable medical device.

13. The method of claim 8, wherein the implantable sensor is at least partially implanted in connective tissue surrounding the shaft of the penis.

14. The method of claim 1, further comprising:
sequentially delivering therapy according to a plurality of therapy parameter values;
emitting the electrical signal from the first electrode during delivery of therapy according to the plurality of therapy parameter values;
detecting the electrical signal via the second electrode during delivery of therapy according to the plurality of therapy parameter values; and
selecting a therapy parameter value based on values of the electrical signal during delivery of therapy according to the plurality of therapy parameter values.

15. A system comprising:
a first electrode implanted within a penis of a patient at a first location that emits an electrical signal;
a second electrode implanted within the penis at a second location that detects the electrical signal; and
an implantable medical device that delivers therapy to the patient based on the detected electrical signal to control penile tumescence.

16. The system of claim 15, wherein the implantable medical device delivers therapy to initiate or sustain an erection.

17. The system of claim 15, wherein the implantable medical device delivers electrical stimulation to the patient.

18. The system of claim 17, wherein the implantable medical device delivers stimulation to at least one of a prostate parasympathetic nerve, a cavernous nerve, a pudendal nerve, or a sacral nerve.

19. The system of claim 15, wherein the implantable medical device delivers therapy based on at least one of a voltage or a current of the detected electrical signal.

20. The system of claim 19, further comprising processing circuitry to determine an impedance based on at least one of the voltage or the current, wherein the implantable medical device delivers therapy based on the impedance.

21. The system of claim 15, wherein at least the second electrode is electrically coupled to the implantable medical device, and the implantable medical device detects the electrical signal via the second electrode.

22. The system of claim 15, further comprising an implantable sensor that is separate from the implantable medical device and electrically coupled to at least the second electrode, wherein the implantable sensor detects the electrical signal.

23. The system of claim 22, wherein the sensor transmits information relating to the detected signal to an external programmer.

24. The system of claim 23, wherein the external programmer controls delivery of stimulation by the implantable medical device based on the information received from the sensor.

25. The system of claim 23, wherein the external programmer presents penile tumescence information to the user based on the information received from the sensor.

26. The system of claim 22, wherein the implantable sensor is at least partially implanted in connective tissue surrounding the shaft of the penis.

27. The system of claim 22, wherein the implantable sensor comprises a housing, one of the first and second electrodes is formed on the housing, and the other of the first and second electrodes is coupled to the implantable sensor by a lead.

28. The system of claim 22, wherein the implantable sensor comprises a first implantable module, the system further comprising a second implantable module that separate from the implantable medical device and the first implantable module, and is electrically coupled to the first electrode.

29. The system of claim 15, further comprising a processor, wherein the implantable medical device sequentially delivers therapy according to a plurality of therapy parameter values, the first electrode emits the electrical signal during delivery of therapy according to the plurality of therapy parameter values, the second electrode detects the electrical signal during delivery of therapy according to the plurality of therapy parameter values, and the processor selects a therapy parameter value based on values of the electrical signal during delivery of therapy according to the plurality of therapy parameter values.

30. A system comprising:
a first electrode implanted within the penis at a first location to emit an electrical signal;
a second electrode implanted within the penis at a second location; and
an implantable sensor comprising a housing implanted within a penis,
wherein the implantable sensor is coupled to at least the second electrode, detects the electrical signal via the second electrode, and comprises telemetry circuitry that wirelessly transmits information relating to the detected electrical signal, and
wherein the information indicates the degree of tumescence of the penis.

31. The system of claim 30, further comprising an implantable medical device that receives the information transmitted from the implantable sensor, and delivers therapy to control penile tumescence based upon the received information.

32. The system of claim 30, further comprising an external programmer that receives the information transmitted from the implantable sensor, and presents penile tumescence information to the user based on the information received from the sensor.

33. The system of claim 30, wherein one of the first and second electrodes is formed on the housing of the implantable sensor, and the other of the first and second electrodes is coupled to the implantable sensor by a lead.

34. The system of claim 30, wherein the implantable sensor comprises a first implantable module, the system further comprising a second implantable module that separate from the implantable medical device and the first implantable module, and is electrically coupled to the first electrode

35. The system of claim 34, further comprising an implantable medical device that delivers therapy to the patient based on the detected electrical signal to control penile tumescence, wherein data is wirelessly transmitted between at least two of the implantable medical device, the first implantable module and the second implantable module, the data including information relating to the detected electrical signal, operating instructions, or other data required to sense and control penile tumescence.
